(19) United States
(12) Patent Application Publication
(10) Pub. No.: US 2012/0046698 A1
(43) Pub. Date: Feb. 23, 2012

(54) METHODS AND DEVICES FOR SPINAL FUSION

(75) Inventors: Eric Kolb, Sandy Hook, CT (US); Naresh Akkarapaka, West Haven, CT (US); John Boxberger, Sandy Hook, CT (US); John Tomich, Wallingford, CT (US)

(73) Assignee: Doctors Research Group, Inc., Southbury, CT (US)

(21) Appl. No.: 12/858,585

(22) Filed: Aug. 18, 2010

(51) Int. Cl. A61B 17/86

(52) U.S. Cl. 606/304

(57) ABSTRACT

A pedicle fastener for implantation during a spinal fusion procedure includes a head and shank, the shank being connectable to the head and extending outwardly therefrom. The shank may include a shielding feature formed therein for accommodating an adhesive augmenting the pedicle fastener and improving implantation strength and stiffness thereof. A method for implanting the pedicle fastener includes forming a hole in a pedicle, applying the adhesive to the hole and sliding the shank of the pedicle fastener into the hole. The adhesive may also be applied to the hole through direct injection, injection through a longitudinal cannula formed in the pedicle fastener or by depositing the adhesive on the shank and inserting the shank into the hole. Polymerization of the adhesive may be accelerated by heating the pedicle fastener.

### FIELD OF THE INVENTION

The present invention relates to methods and devices for use in spinal fusion and, more particularly, to bone fasteners and methods of implantation thereof.

### BACKGROUND OF THE INVENTION

There are many situations in which bones or bone fragments are fused, including fractures, joint degeneration, abnormal bone growth, infection and the like. For example, circumstances requiring spinal fusion include degenerative disc disease, spinal disc herniation, discogenic pain, spinal tumors, vertebral fractures, scoliosis, kyphosis, spondylolisthesis, spondylosis, Posterior Rami Syndrome, other degenerative spinal diseases, and other conditions that result in instability of the spine. In many situations, bone screws are used in orthopedic surgery to stabilize the bones or to affix stabilizing equipment thereto.

For instance, during spinal fusion, pedicle screws are often implanted to correct, or to aid in correcting, the spinal instability. For example, pedicle screws may be implanted to assist spinal fusion by holding unstable bone structures together to immobilize a portion of the spine. The pedicle screws are typically implanted to affix stabilizing equipment such as rods or plates to the spine.

Typically, the pedicle screw is inserted directly into the patient's bone to form a screw purchase. However, in some patient populations, the pedicle screws loosen and fail after installation, particularly for patients with osteoporosis or where the pedicle screw was installed to replace a previously failed pedicle screw that already stripped the screw hole. Therefore, solutions have been developed to repair and/or discourage these screw failures.

One known solution for improving the screw purchase is to increase the size of the pedicle screw. However, in many instances, the size and/or shape of the patient's pedicle may limit the size of the pedicle screw, thereby making it impossible to increase the size thereof.

A second known solution for discouraging failure of the screw purchase is to provide a geometric feature on the pedicle screw that interacts with the bone, such as an expanding screw tip. However, these geometric features may compromise implant strength and may be difficult to revise in the future, if necessary.

A third known solution for discouraging screw purchase failure is to augment the screw purchase with PMMA (polymethyl methacrylate). However, the stiffness of PMMA does not match that of the surrounding bone, which can lead to additional stress at the screw purchase. Additionally, the time before the PMMA sets, during which it may be implanted and/or worked, is relatively small, providing a surgeon with a very limited window in which the augmented pedicle screw must be inserted. In the event of a screw purchase failure or implantation error, PMMA is also difficult to rework, due to its rigidity and brittleness. Additionally, PMMA is not inherently adhesive to either bone or metal and, therefore, may not form a long lasting bone-PMMA or PMMA-screw interface. The act of screwing the pedicle screw into the patient's bone may also scrape the PMMA off of the threads of the pedicle screw, thereby further compromising the screw purchase.

A fourth solution for discouraging failure of the screw purchase is to augment the screw purchase with mineral cement such as calcium phosphate. However, mineral cements have low tensile strength and may resorb before adequate bone ingrowth is achieved around the pedicle screw. Additionally, mineral cement, like PMMA, is not inherently adhesive to either bone or metal and, therefore, may not form a long lasting interface.

Accordingly, there is a need for pedicle fasteners and methods and systems of implantation thereof that provide more workability and easier implantation. Additionally, there is a need for a pedicle fastener implantation system that allows for augmentation of pedicle fasteners with adhesive that provides adhesion with the pedicle fasteners and the surrounding bone and that approximates the strength and stiffness of natural bone to provide mechanically stable augmentation of the pedicle fasteners. Although the present invention will be described in terms of pedicle fasteners for simplicity, it should be understood by those skilled in the art that the methods and devices of the present invention are equally applicable to other bone fasteners of varying shapes and sizes.

### SUMMARY OF THE INVENTION

According to the present invention, a pedicle fastener for implantation during a spinal fusion procedure includes a head and shank. The shank is connectable to the head and extends outwardly therefrom. The shank may include a shielding feature formed therein for accommodating an adhesive augmenting the pedicle fastener for improving implantation strength and stiffness thereof. The adhesive may be a biocompatible polymeric material and, preferably, is biocompatible polyurethane. In some embodiments, the adhesive and the pedicle fastener may be provided together as a spinal fusion kit.

According to the present invention, the shielding feature may take a variety of forms including annular grooves, longitudinal or spiral flutes, slots, channels, threads and/or combinations thereof. The shank may also include a reverse taper at its tip for centering the pedicle fastener. In some embodiments, attachable features may be provided for, or pre-attached to, the pedicle fastener for controlling adhesive flow of the adhesive and/or for filling the pedicle hole. For instance, the pedicle fastener may include a restrictive disc attached to the shank. The pedicle fastener may include a surface coating formed on at least a portion of the shank for improving adhesion thereto. In some embodiments, the pedicle fastener may include a mesh sleeve surrounding the shank for controlling adhesive flow.

According to some embodiments of the present invention, the pedicle fastener may also include a longitudinal cannula extending at least partially through the head or shank. The longitudinal cannula may be adapted to allow cleaning and/or drying of the implant site after the pedicle fastener has been inserted therein. The longitudinal cannula may also allow injection of the adhesive through the pedicle fastener into the implant site after the pedicle fastener has been inserted therein. In some embodiments, the pedicle fastener may also include fenestrations connecting the longitudinal cannula with the shielding features to deliver adhesive thereto.

According to the present invention, the pedicle fastener may include a heating feature for applying a heat source to the pedicle fastener to accelerate polymerization. In some embodiments, the heating feature may be the longitudinal cannula, which may accept a heating element therein for accelerating polymerization of the adhesive. In other embodiments, the heating feature may include a removable extension extending from the head of the pedicle fastener. In some embodiments, the pedicle fastener may be provided with frozen adhesive provided thereon such that the frozen adhesive thaws and polymerizes after implantation. In some embodiments, the frozen adhesive may thaw and polymerize due to natural body heat, while in other embodiments, polymerization of the frozen adhesive may advantageously be accelerated using the heating features discussed above.

According to the present invention, a method for implanting a pedicle fastener includes forming a hole in a pedicle having a diameter sufficient to surround the shank of the pedicle fastener, applying an adhesive to the hole and sliding the shank of the pedicle fastener into the hole. The method may also include forming the adhesive by mixing a prepolymer component, a polyol component and a filler material.

According to some embodiments of the present invention, the adhesive may be deposited on the shank of the pedicle fastener such that the adhesive is applied to the hole when the shank is slid therein. When depositing the adhesive on the shank, it may be desirable to apply the adhesive to at least one shielding feature formed in the shank so that the adhesive is not scraped off of the shank upon insertion into the hole.

According to the present invention, the adhesive may also be applied to the hole by injecting the adhesive through a syringe. In some embodiments, the adhesive may be injected through a longitudinal cannula formed in the pedicle fastener after the pedicle fastener has been inserted into the pedicle hole.

According to some embodiments of the present invention, the method may also include heating the pedicle fastener to accelerate polymerization of the adhesive. In some embodiments, an electric heating element may be connected to the head of the pedicle fastener. In other embodiments, a heating element may be inserted into a longitudinal cannula extending through at least a portion of the pedicle fastener. In some embodiments, the pedicle fastener may be heated under a heat lamp or may be preheated prior to insertion into the pedicle hole.

These and other objects, features and advantages of the present invention will become apparent in light of the following detailed description of non-limiting embodiments, with reference to the accompanying drawings.

### BRIEF DESCRIPTION OF THE DRAWINGS

FIG. 1 is a side perspective view of a pedicle fastener according to an embodiment of the present invention;

FIG. 2 is a perspective view of an embodiment for forming an adhesive for augmenting the pedicle fastener of FIG. 1;

FIG. 3 is a perspective view of a kit for implanting the pedicle fastener of FIG. 1;

FIG. 4 is a cross-sectional view of the pedicle fastener of FIG. 1 immediately after implantation;

FIG. 5 is a cross-sectional view of the implanted pedicle faster of FIG. 4 after curing of the adhesive;

FIG. 6 is a cross-sectional view of adhesive being applied to a bone hole according to another embodiment of the present invention;

FIG. 7 is a cross-sectional view of the bone hole of FIG. 6 with a pedicle fastener inserted therein;

FIG. 8 is a cross-sectional view of an implanted pedicle fastener according to another embodiment of the present invention;

FIG. 9 is a side perspective view of a pedicle fastener according to a further embodiment of the present invention;

FIG. 10 is a side perspective view of a pedicle fastener according to a further embodiment of the present invention;

FIG. 11 is a side perspective view of a pedicle fastener according to a further embodiment of the present invention;

FIG. 12 is a cross-sectional view of the pedicle fastener of FIG. 11;

FIG. 13 is a cross-sectional view of adhesive being applied to a pedicle fastener according to another embodiment of the present invention;

FIG. 14 is a side perspective view of the pedicle fastener of FIG. 1 according to yet another embodiment of the present invention;

FIG. 15 is a cross-sectional view of a further embodiment of a pedicle fastener according to the present invention;

FIG. 16 is a partial cross-sectional view of another embodiment of a pedicle fastener according to the present invention;

FIG. 17 is a side perspective view of the pedicle fastener of FIG. 1 according to yet another embodiment of the present invention;

FIG. 18 is a cross-sectional view of a further embodiment of a pedicle fastener according to the present invention;

FIG. 19 is a cross-sectional view of another embodiment of a pedicle fastener according to the present invention;

FIG. 20 is a cross-sectional view of yet another embodiment of a pedicle fastener according to the present invention;

FIG. 21 is a cross-sectional view of an embodiment for accelerated polymerization of adhesive according to the present invention; and

FIG. 22 is a cross-sectional view of another embodiment for accelerated polymerization of adhesive according to the present invention.

### DETAILED DESCRIPTION OF A PREFERRED EMBODIMENT

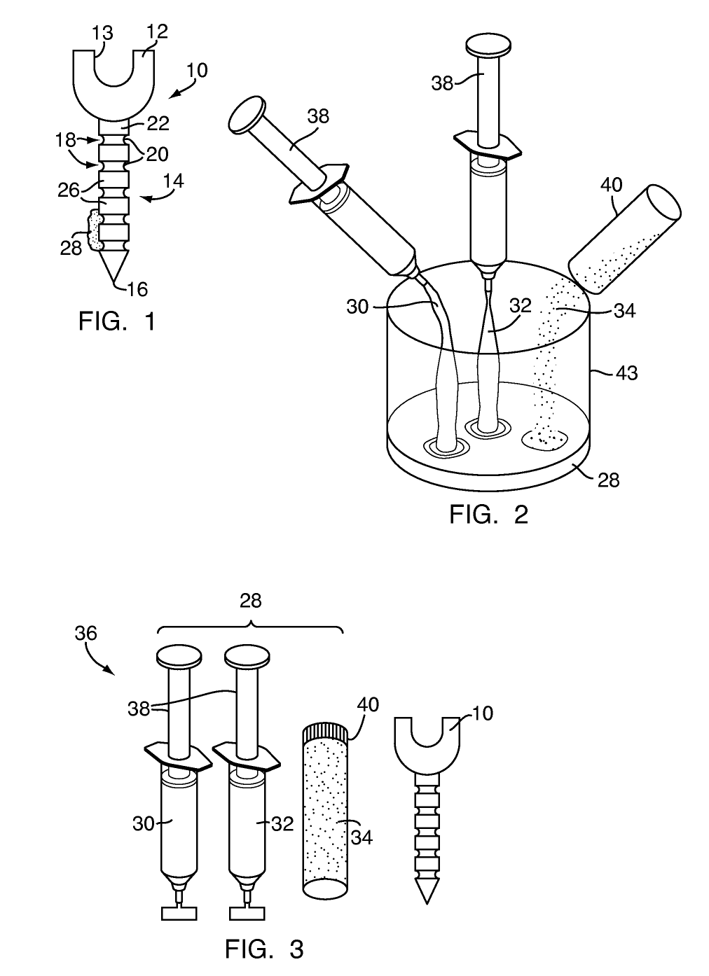

Referring to FIG. 1, a pedicle fastener 10 includes a head 12 and a shank 14 extending outwardly from the head 12 to a shank tip 16. The head 12 may be similar to known pedicle screw heads, including at least one suitable connection feature 13 for rods, plates or the like. The shank 14 has at least one shielding feature 18 formed therein. For example, the shielding features 18 may be a plurality of annular grooves 20 formed in an outer surface 22 of the shank 14 and extending around a circumference thereof. The annular grooves 20 are separated by ridges 26 also extending around the circumference of the shank 14. In some embodiments, the annular grooves 20 and the ridges 26 may include smooth transitions therebetween, providing the outer surface 22 with an undulating sinusoidal-type profile.

Although shown as a single unit, in some embodiments, the pedicle fastener 10 may be part of a spinal fusion device (not shown) including the pedicle fastener 10, a rod (not shown) and a locking member (not shown). Like known pedicle screws, the head 12 and the shank 14 may be swivelable or polyaxial and, in some embodiments, may be able to be connected and disconnected from one another, depending upon the intended application.

The annular grooves 20 and ridges 26 allow the shank 14 to be augmented by an adhesive 28 to improve fixation of the pedicle fastener 10. In particular, the adhesive 28 may be applied to the shank 14 within the annular grooves 20, where it will be protected during installation by the ridges 26, as will be discussed in greater detail below.

The adhesive 28 is preferably a reactive biocompatible polymeric material, which has adhesive characteristics. Additionally, the adhesive 28 is preferably osteoconductive when used in medical procedures. An example of one suitable reactive biocompatible polymeric material for the adhesive 28 is the KRYPTONITE™ bone cement product, available from DOCTORS RESEARCH GROUP, INC. of Southbury, Conn., which is described in U.S. patent application Ser. No. 11/089,489, which is hereby incorporated by reference in its entirety.

Referring to FIG. 2, in some embodiments, the adhesive 28 may be formed by combining a prepolymer component 30 and a polyol component 32, along with an optional filler material 34, and permitting the combination to react to form the adhesive 28.

The prepolymer component 30 for forming the adhesive 28 includes prepolymer molecules formed by reacting diisocyanate with polyol. The prepolymer component 30 may be a true prepolymer, formed with a two to one ratio of diisocyanate to polyol, or the prepolymer component 30 may be a quasi-prepolymer, formed with a ratio of diisocyanate to polyol in excess of two to one. As will be understood by those skilled in the art, a broad variety of diisocyanates and polyols may be suitable for use in the prepolymer component 30 and the adhesive 28 of the present invention. Both aromatic and aliphatic diisocyanates may be used to form the prepolymer component 30 of the present invention. The polyol used to form the prepolymer component 30 may be the same as or different than the polyol of the polyol component 32. Additionally, the polyol used to form the prepolymer component 30 may be a blend of different polyols to achieve desired properties. Various polyols suitable for synthesis with the diisocyanate will be discussed in greater detail below.

The polyol component 32 for forming the adhesive 28 may include naturally occurring polyols and biocompatible, synthetic polyols, and mixtures thereof to achieve desired properties in the adhesive 28. The polyol component 32 preferably also includes a catalyst for controlling and/or reducing the time required for polymerization of the adhesive 28. Additionally, the polyol component 32 may include water, which is known to react with diisocyanate to produce carbon dioxide. Thus, the water may be provided to react with the diisocyanate to generate a sufficient amount of carbon dioxide to impart a degree of porosity to the adhesive 28. Alternatively, rather than including water in the polyol component 32, moisture from the atmosphere or moisture included in the optional filler material 34 may impart the degree of porosity to the adhesive 28. Additionally, in instances where moisture is provided from the atmosphere or within the optional filler material 34, it may be desirable to dry the polyols to provide improved control over the amount of carbon dioxide produced and, therefore, the degree of porosity imparted to the adhesive 28.

The optional filler material 34 for forming the adhesive 28 may include, but is not limited to, calcium carbonate, bone (e.g., demineralized bone, allograft bone, and/or autogenous bone), calcium phosphate, calcium pyrophosphate, hydroxyapatite, poly methyl methacrylate, glass-ionomer, calcium sulfate, tricalcium phosphate (e.g., beta tricalcium phosphate), or any combination thereof, or the like. In certain embodiments, the filler material 34 may be chosen so as to impart a desired degree of porosity to the adhesive 28. For example, the filler material 34 may include water for reacting with the diisocyanate of the prepolymer component 30 to generate carbon dioxide and impart the porosity to the adhesive 28. The filler material 34 may also be present in the adhesive 28 in an amount sufficient to modify the adhesive's mechanical properties (e.g., compressive strength, compressive modulus, Young's Modulus of Elasticity, flexural strength, and the like). The filler material 34 may also comprise calcium carbonate and, in certain of these embodiments, the filler material 34 may comprise calcium carbonate in an amount sufficient to provide free calcium to a body of a mammal and enhance osteoconductivity. In some embodiments, the filler material 34 may be a material that allows visualization under radiographic imaging, such as barium sulfate, so that the condition of the adhesive 28 may be determined in situ through non-invasive imaging procedures.

Although the adhesive 28 may be formed with a variety of compositions to achieve desired properties, preferably, the adhesive 28 is a biocompatible polyurethane material, wherein the prepolymer component 30 includes aromatic pMDI diiscyanates synthesized with polyols derived from castor oil. The polyol component 32 preferably also includes polyols derived from castor oil and a small percentage of catalyst. The optional filler material 34 is preferably calcium carbonate powder, at a concentration of thirty percent (30%) by weight, with approximately ninety percent (90%) of the powdered particles being less than ten microns (10 μm) in diameter.

The adhesive 28 is initially prepared in a liquid state when the prepolymer component 30, polyol component 32 and optional filler material 34 are combined. The adhesive 28 is chemically adhesive in this liquid state. As the adhesive 28 cures, it passes through a taffy-like state, in which the adhesive 28 is still chemically adhesive and is also easily malleable and may be shaped and sculpted. The biocompatible polymeric material then passes into a putty-like state in which the material's adhesive properties are reduced and the material is easily malleable and may be shaped and sculpted. The adhesive 28 then cures into a final solid state. As the adhesive 28 cures, it becomes more viscous and less adhesive, i.e., the adhesive 28 loses tackiness, which, as used herein, is defined as the ability of the adhesive 28 to be slightly adhesive or gummy to the touch or to adhere to a dry surgical instrument, for example, a freer. Additionally, the adhesive 28 expands as it cures from the liquid state to the final solid state. Since the adhesive properties of the adhesive 28 are greatest when the material is in the liquid or taffy-like state, the adhesive 28 is preferably in either the liquid state or the taffy-like state when used to augment the pedicle fastener 10, shown in FIG.

Referring to FIG. 3, a kit 36 for implanting pedicle fasteners 10 includes at least one pedicle fastener 10, as well as the prepolymer component 30 and polyol component 32 for forming the adhesive 28. The kit 36 may also include the optional filler material 34 for forming the adhesive 28, if desired. The prepolymer component 30, polyol component 32 and optional filler material 34 are each held in a suitable container such as syringes 38, canister 40 or the like.

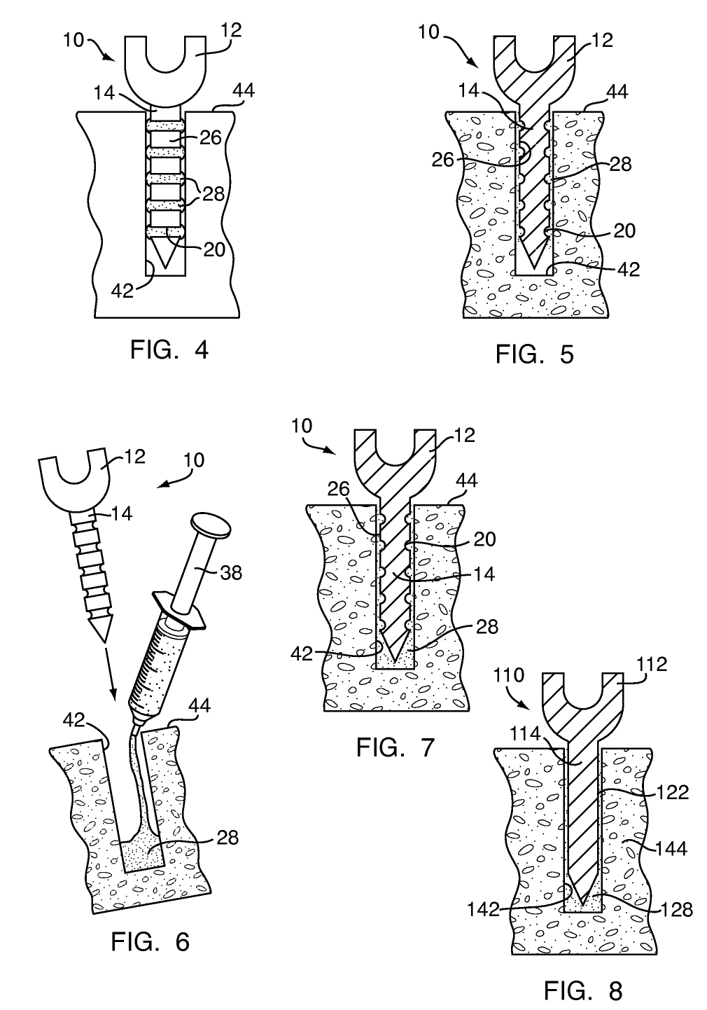

Referring to FIG. 4, in operation, during spinal fusion a surgeon first prepares a hole 42 in bone 44. For example, the surgeon may drill hole 42 or, in the case of a revision surgery, the hole 42 may be preexisting and may simply be cleaned or resized by the surgeon. Preferably, the hole 42 has a diameter that is as large as or larger than a diameter of the ridges 26 of the shank 14. Preparation of the hole 42 preferably also includes cleaning and or drying the bone 44 forming the inner surface of the hole 42 to achieve a clean dry surface with which the adhesive 28 may bond. For instance, in some embodiments, a brush (not shown), such as a test tube brush, may be inserted into the hole 42 to clean the hole of debris. Additionally, or in the alternative, the hole 42 may be cleaned with a cleaning fluid, for example, by rinsing with a liquid (e.g. sterile saline) or by drying the bone 44 of the hole 42 with a gas such as compressed air or carbon dioxide through a commercial lavage system. In some embodiments, cleaning of the hole 42 may be achieved by applying a hemostatic agent to the hole 42, for example, by applying the hemostatic agent on a brush used to clean the hole 42.

As seen in FIG. 2, the surgeon may mix the prepolymer component 30, polyol component 32 and optional filler material 34 from the kit 36, shown in FIG. 3, to form the adhesive 28. Referring back to FIG. 4, the surgeon may then apply the adhesive 28 to the shank 14 and, in particular, to the annular grooves 20 of the shank 14. Preferably, the adhesive 28 is substantially viscous when applied to the shank 14. The surgeon may then install the pedicle fastener 10 by sliding the shank 14 into the hole 42. While the shank 14 is slid into the hole 42, the adhesive 28 is shielded within the annular grooves 20 by the ridges 26 and, therefore, is not scraped out of the annular grooves 20 and off of the shank 14 by the surface of the hole 44. The pedicle fastener 10 is then maintained in position within the hole 42 while the adhesive 28 cures.

Referring to FIG. 5, as the adhesive 28 cures, it expands outward from the annular grooves 20 and into the hole 42, contacting the bone 44 and expanding into the porous structure of the bone 44. Once the adhesive 28 has cured, it provides strong chemical adhesion between the pedicle fastener 10 and the bone 44 due to the adhesive characteristics of the adhesive 28. Additionally, the adhesive 28 provides strong mechanical adhesion since the adhesive 28 cures at least partially within the annular grooves 20 and also expands at least partially into the porous structure of the bone 44 and cures therein. Thus, even though the pedicle fastener 10 is not threadedly engaged with the bone 44, the pedicle fastener 10 has a strong mechanical bond therewith. Additionally, the adhesive provides a chemical bond between the pedicle fastener 10 and the bone 44 since the adhesive 28 was not scraped out of the annular grooves 20 by the bone 44 due to a threaded engagement.

In addition to the chemical and mechanical bonds provided by the adhesive 28, the adhesive 28 may also be formulated to provide strength and stiffness similar to that of bone 44, thereby reducing stress concentrations at the spinal fusion site. Additionally, the adhesive 28 may provide an osteoconductive structure to promote ingrowth of bone 44 into the adhesive 28 and hole 42, thereby providing for improved patient recovery.

Referring to FIG. 6, in some embodiments of the present invention, after preparation of the hole 42 as discussed above, the hole 42 may be filled with adhesive 28 prior to insertion of the pedicle fastener 10, shown in FIG. 1, therein. For example, the adhesive 28 may be injected into the hole 42 through syringe 38 or the like. Once the adhesive 28 has been injected into the hole 42, the pedicle fastener 10 may be inserted therein. The shank 14 of the pedicle fastener 10 may have adhesive 28 applied thereto, for example, within the annular grooves 20 as discussed above or may be inserted without any adhesive 28 applied thereto, since adhesive 28 is already within the hole 42.

Referring to FIG. 7, as the pedicle fastener 10 is slid into the hole 42, the shank 14 displaces the adhesive 28 up and around the shank 14 within the hole 42. The pedicle fastener 10 is then maintained in position within the hole 42 while the adhesive 28 cures. As the adhesive 28 cures, it expands, filling the annular grooves 20 and at least partially invading the porous structure of the bone 44. As discussed above, once the adhesive 28 has cured, it provides strong chemical adhesion between the pedicle fastener 10 and the bone 44 due to the adhesive characteristics of the adhesive 28. Additionally, the adhesive 28 provides strong mechanical adhesion since the adhesive 28 cures at least partially within the annular grooves 20 and also expands at least partially into the porous structure of the bone 44 and cures therein.

Referring to FIG. 8, wherein like numerals represent like elements, in some embodiments, particularly those where the adhesive 128 is applied to the hole 142 prior to insertion of the pedicle fastener 110, the outer surface 122 of the shank 114 may be substantially smooth, rather than having shielding features 18, shown in FIG. 1, formed therein. The shank 114 may have a circular cross-section to substantially conform to the shape of the hole 142 or the shape may have an acircular cross-section, e.g. an elliptical or oval cross-section, to provide space within the hole 142 between the bone 144 and the shank 114 for the adhesive 128 be displaced and expand. Acircular cross-sections may also be beneficial in that they may provide greater mechanical strength and/or stiffness in some planes than other planes, thereby allowing for optimization of both the mechanical properties and the adhesive potential of the pedicle fastener 110. Additionally, pedicle fasteners 110 with acircular cross sections may also be beneficial since pedicle bones are typically acircular in cross section, such that a corresponding pedicle fastener geometry may provide a superior fit. Pedicle fasteners 110 with acircular cross sections may be particularly advantageous for use with pedicles devoid of cancellous bone, i.e. through osteoporosis or iatrogenic causes, since the pedicle fasteners 110 may be implanted within the voids formed by the absence of the cancellous bone and may be better shaped to fill said voids than other pedicle fasteners.

Since the adhesive 128 is applied to the hole 142 rather than the shank 114 prior to insertion of the shank 114 into the hole 142, the shielding features 18, shown in FIG. 1, are not necessary to protect the adhesive 128 from being scraped off of the shank 114 during insertion. The smooth outer surface 122 may more easily allow the adhesive 128 to be displaced upward around the shank 114 when the pedicle fastener 110 is implanted and may provide a broad contact surface with which the adhesive 128 may chemically bond. Although the total surface area of the shank 114 with the smooth outer surface 122 will be less than the shank 14 having shielding features 18, shown in FIG. 1, the realized adhesive 128 to outer surface 122 contact area may be greater with the smooth outer surface 122, since some of the annular grooves 20, shown in FIG. 1, may not be completely filled by the displaced adhesive 28, shown in FIG. 1, when the pedicle fastener 10, shown in FIG. 1, is implanted. Thus, the pedicle fastener 110 with the smooth outer surface 122 may provide for increased chemical adhesion with the adhesive 128.

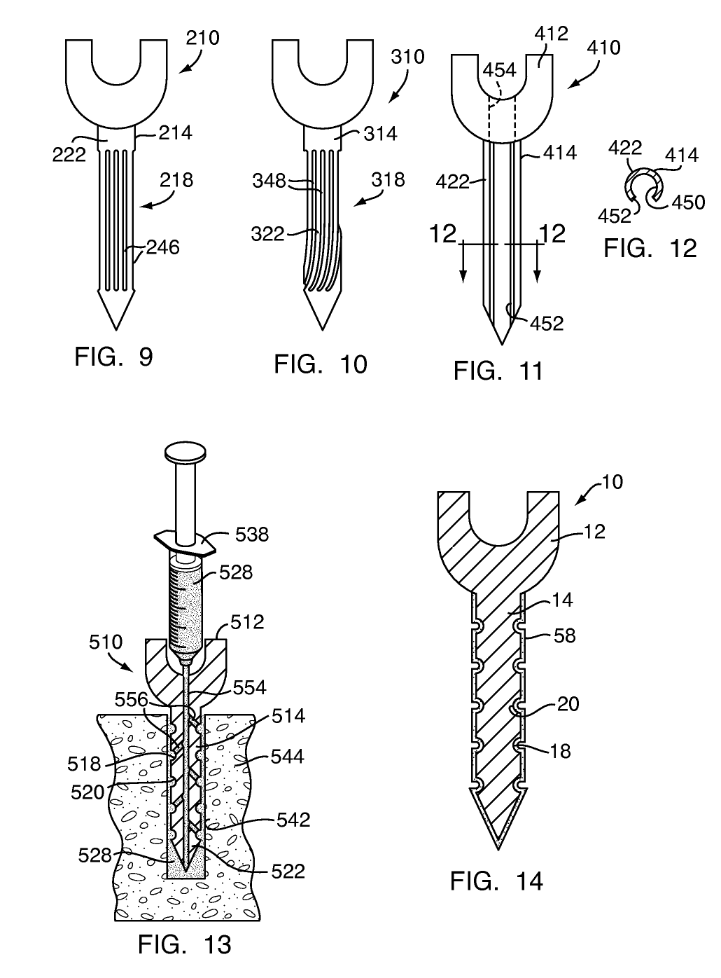

Referring to FIG. 9, in some embodiments, the pedicle fastener 210 may include shielding features 218 that are flutes 246 extending longitudinally along at least a portion of the shank 214. In operation, the pedicle fastener 210 is preferably implanted after adhesive 28, shown in FIG. 6, has been injected into the hole 42, shown in FIG. 6. In particular, as the shank 214 is slid into the hole 42, shown in FIG. 6, the flutes 246 will facilitate displacement of the adhesive 28, shown in FIG. 6, upward around the outer surface 22 of the shank 214. Additionally, as the adhesive 28, shown in FIG. 6, cures, the flutes 246 may provide an avenue for expansion of the adhesive 28, shown in FIG. 6. The flutes 246 also provide the outer surface 222 of the shank 214 with an increased surface area with which the adhesive 28, shown in FIG. 6, may bond. Although the various embodiments of shielding features 218 have been described separately herein for simplicity, it should be understood by those skilled in the art that the embodiments may be combined to achieve desired adhesive flow and bonding characteristics. For example, the pedicle fastener 210 may include both flutes 246 and annular grooves 20, shown in FIG. 1, interconnected therewith to provide the outer surface 222 of the pedicle fastener 210 with a further increased surface area, while still providing flutes 246 for improved adhesive flow therethrough.

Referring to FIG. 10, in another embodiment of the present invention, the pedicle fastener 310 may be provided with shielding features 318 that include spiral flutes 348, having both a longitudinal component and a circumferential component. As with the embodiments discussed above, the pedicle fastener 310 provides an increased surface area with which the adhesive may bond while still providing a path for adhesive displacement and expansion along the shank 314. Due to the longitudinal and circumferential components of the spiral flutes 248, this embodiment may be particularly beneficial where the pedicle fastener 310 is not only pushing into the hole 42, shown in FIG. 6, but also rotated slightly while being inserted.

Referring to FIGS. 11 and 12, in some embodiments, the pedicle fastener 410 may include shank 414 bounding an inner longitudinal channel 450 that is substantially coaxial with the shank 414, similar to a rolled pin. A longitudinal slot 452 passes through the shank 414 and is in communication with the longitudinal channel 450. The longitudinal slot 452 preferably extends substantially the entire length of the shank 414. This embodiment further increases the surface area of the shank 414 with which the adhesive 28, shown in FIG. 6, can bond, since the adhesive 28, shown in FIG. 6, may fill and bond with the longitudinal channel 450 in addition to the outer surface 422.

Referring back to FIG. 6, thus far, the present invention has been described as having adhesive 28 applied to at least one of the shank 14 or the hole 42 prior to insertion of the pedicle fastener 10. However, referring to FIG. 13, in some embodiments, the pedicle fastener 510 may be inserted into the hole 542 prior to application of the adhesive 528. The pedicle fastener 510 includes head 512 and shank 514, which are substantially similar to those discussed above. The outer surface 522 of the shank 514 may include shielding features 518, such as the annular grooves 520 as shown, or may be substantially smooth as discussed above. The pedicle fastener 510 also includes an internal longitudinal cannula 554 extending through the head 512 and at least partially through the shank 514. Connected to the longitudinal cannula 554 are a plurality of fenestrations 556 that extend outwardly through the outer surface 522. Preferably, the fenestrations 556 extend into the shielding features 518, if present.

In operation, the surgeon may push the pedicle fastener 510 into the hole 542 in substantially the same manner discussed above. In some embodiments, the surgeon may then spray a cleaning fluid, which may be a liquid (e.g. sterile saline) or a gas (e.g. compressed air or carbon dioxide from a lavage system), into the longitudinal cannula 554 through the head 512. The cleaning fluid passes through the fenestrations 556 and is expelled into the hole 542, thereby cleaning and/or drying the bone 544 prior to injection of the adhesive 528. Alternatively, the surgeon may simply prepare and clean the hole 542 prior to insertion of the pedicle fastener 510 therein as discussed above. Using a syringe 538 or similar injection tool, the surgeon may then inject the adhesive 528 through the head 512 and into the longitudinal cannula 554. The adhesive 528 passes through the fenestrations 556, into the annular grooves 520 and into contact with the bone 544. The adhesive 528 is then allowed to cure to form a bond between the pedicle fastener 510 and the bone 544 as discussed above.

Although described as having the adhesive 528 injected into the longitudinal cannula 554 after inserting the pedicle fastener 510 into the hole 542, in some embodiments, the adhesive 528 may first be injected into the hole 542 as discussed above prior to insertion of the pedicle fastener 510. Then, once the pedicle fastener 510 has been inserted into the hole 542, a vacuum may be connected to the longitudinal cannula 554 to draw adhesive against the shank 514 and into the fenestrations 556 to provide improved adhesion therewith. Additionally, although described in connection with the embodiment having annular grooves 520, the longitudinal cannula 554 and fenestrations 556, or variations thereof, may be implemented in the other embodiments discussed herein. For example, referring back to FIG. 11, the longitudinal cannula 454 may extend through the head 412 and be in communication with the longitudinal channel 450 extending through the shank 414, thereby allowing adhesive 528, shown in FIG. 13, to be injected into the longitudinal channel 450 after insertion of the pedicle fastener 410 into hole 542, shown in FIG. 13.

Referring to FIG. 14, in another embodiment of the present invention, the pedicle fastener 10 may include a surface coating 58 to further improve adhesion between the pedicle fastener 10 and the adhesive 28, shown in FIG. 6. The surface coating 58 may be, for example, formed from the same biocompatible polymeric material as the adhesive 28, shown in FIG. 6. Preferably, once cured on the shank 14, the surface coating 58 is machined to remove any nonporous skim coat that could inhibit a strong bond with the adhesive 28. In some embodiments, the surface coating 58 may be hydrophobic such that it forces water and blood away from the surface to allow better wetting of the liquid adhesive 28 on the pedicle fastener 10. Although shown with annular grooves 20, the surface coating 58 may be applied to the various other shielding features 18 discussed herein or may be applied to the substantially smooth shank 114, shown in FIG. 8.

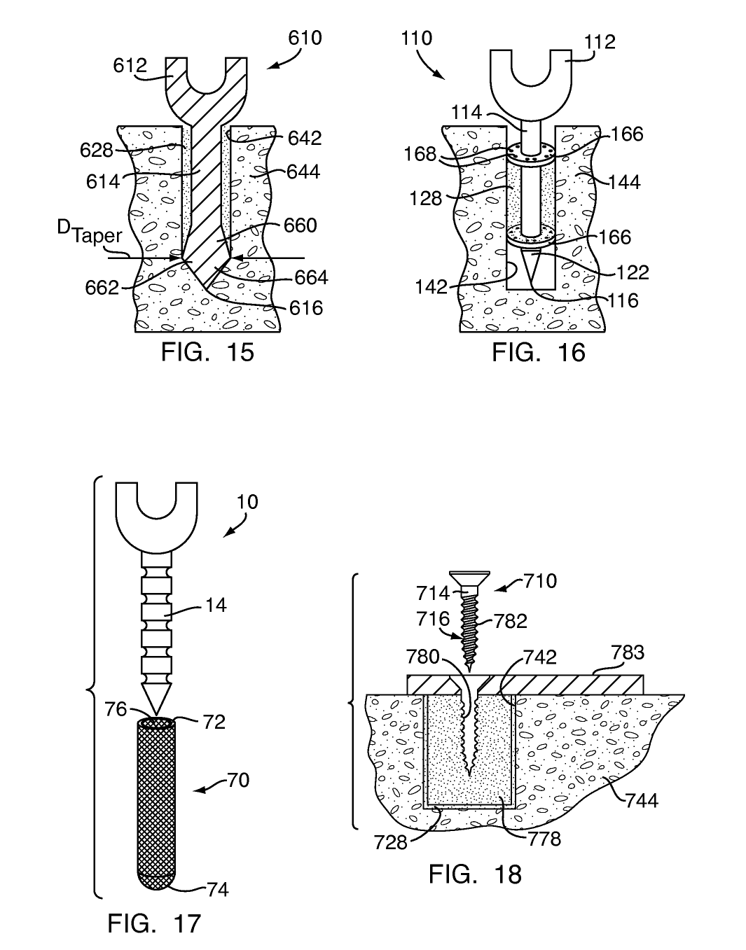

Referring to FIG. 15, in some embodiments, the shank tip 616 may include a reverse taper 660 such that a diameter D_{Taper} of the distal end 662 of the reverse taper 660 is greater than the diameter of the shank 614. The larger diameter D_{Taper} acts to center the fastener within the hole 642 and provides a substantially uniform gap between the shank 614 and the bone 644 to be filled by adhesive 628. In some embodiments, the reverse taper 660 may not extend around the entire circumference of the shank tip 616, but rather may include three or more reverse taper features radiating from the shank 614 to form an effective (circumscribed) diameter D_{Taper} that still centers the fastener within the hole 642. This embodiment may be particularly beneficial, as the space between the reverse taper features may allow for the passage of adhesive 628 therebetween. In some embodiments, the shank tip 616 with the reverse taper 660 may also include a self-drilling tip 664 at the distal end 662 of the reverse taper 660 to allow insertion of the pedicle fastener 610 without additional instrumentation.

Referring to FIG. 16, in another embodiment of the present invention, the pedicle fastener 110 may have restrictive discs 166 connected thereto at two different longitudinal locations along the shank 114. Preferably, one restrictive disc 166 is connected proximate to the shank tip 116 and the second restrictive disc 166 is connected proximate to the head 112. The restrictive discs 166 are formed to be substantially the same diameter as the hole 142. Thus, the restrictive discs 166 surround the outer surface 122 of the shank 114, trapping the adhesive 128 therebetween. Locating one of the restrictive discs 166 proximate to the shank tip 116 may be particularly advantageous as it resists adhesive flow out of the higher density pedicle region and into the vertebral body. In some embodiments, the restrictive discs 166 may have one or more secondary holes 168 to allow a measured amount of adhesive 128 to flow past the restrictive discs 166, for example to allow a predetermined amount of pressure relief as the adhesive 128 expands during polymerization, while still preventing excessive flow of the adhesive 128. The restrictive discs 166 may be provided pre-attached to the pedicle fastener 110 or may me provided as part of the kit 36, shown in FIG. 3, to be attached during implantation. Although shown in connection with the embodiment having a smooth outer surface 122, the restrictive discs 166 may be applied to the other various embodiments discussed herein.

Referring to FIG. 17, in another embodiment of the present invention, a mesh sleeve 70 is provided for accommodating at least a portion of the shank 14 of the pedicle fastener 10. The mesh sleeve has a generally cylindrical shape extending longitudinally from a first end 72 to a second end 74. The first end 72 has a first opening 76 formed therein, while the second end 74 may be either open or closed as desired. The mesh sleeve 70 is preferably formed from metal and is relatively rigid. However, in other embodiments, a more pliable mesh sleeve 70 may be formed from a polymer or the like. The mesh sleeve 70 may be provided as part of the kit 36, shown in FIG. 3.

In operation, the mesh sleeve 70 is pressed into the hole 42, shown in FIG. 6, preferably after preparation of the hole 42, shown in FIG. 6, as discussed above. The mesh structure of the mesh sleeve 70 may advantageously provide for a slight expansion in the longitudinal direction, while simultaneously providing a slight contraction in the diameter of the mesh sleeve 70, which may ease insertion of the mesh sleeve 70 into the hole 42, shown in FIG. 6. Alternatively, if a greater contraction in the diameter is necessary, the mesh sleeve 70 may include a longitudinal slit (not shown) extending from the first end 72 to the second end 74 to allow the mesh sleeve 70 to curl upon itself to reduce the diameter. Once the mesh sleeve 70 has been inserted in the hole 42, shown in FIG. 6, adhesive 28, also shown in FIG. 6, may be injected into the mesh sleeve 70 through the first opening 76. The pedicle fastener 10 may then be pushed into the mesh sleeve 70 through the first opening 76. The pedicle fastener 10 displaces the adhesive 28, shown in FIG. 6, into and through the mesh sleeve 70, where the adhesive 28, shown in FIG. 6, interdigitates with the surrounding bone 44, shown in FIG. 6. The mesh structure of the mesh sleeve 70 holds the adhesive 28, shown in FIG. 6, in place, inhibiting it from inadvertently flowing down into the hole 42, shown in FIG. 6, in the pedicle. Additionally, migration of the adhesive 28, shown in FIG. 6, may be further inhibited if the second end 74 of the mesh sleeve is closed as discussed above.

The mesh structure of the mesh sleeve 70 may be selected to have a mesh size, i.e. the size of the openings of the mesh structure, and mesh pattern, i.e. shape and/or style of the mesh structure, to manage and/or control migration of the adhesive 28, shown in FIG. 6, through the mesh sleeve 70. For instance, in some embodiments, the mesh structure may include approximately square openings with a mesh size having approximate dimensions of less than two millimeters (2 mm) by two millimeters (2 mm). More preferably, the mesh size has approximate dimensions of less than one millimeter (1 mm) by one millimeter (1 mm).

Although the mesh sleeve 70 has been discussed as being provided separately from the pedicle fastener 10, in some embodiments, the mesh sleeve 70 may be connected to the pedicle fastener 10, for example by welding of the like. Similar to the embodiments discussed above, in the pre-connected embodiment of the mesh sleeve 70, the adhesive 28, shown in FIG. 6, may be injected in the hole 42, shown in FIG. 6, prior to simultaneous insertion of the mesh sleeve 70 and the pedicle fastener 10. Alternatively, the adhesive 28, shown in FIG. 6, may be applied directly to the mesh sleeve 70, preferably being pressed into the mesh structure, and then the mesh sleeve 70 and the pedicle fastener 10 may be simultaneously inserted into the hole 42, shown in FIG. 6.

Referring to FIG. 18, in another embodiment of the present invention, an insert 778 may be provided for filling the hole 742, particularly if the hole 742 is relatively large. The insert 778 may have a pre-threaded female thread pattern 780 formed therein for accommodating the pedicle fastener 710. In this embodiment, the pedicle fastener 710 may be a pedicle screw having shielding features 716 in the form of a thread pattern 782 that matches the female thread pattern 780 of the insert 778. In other embodiments, rather than the pre-threaded female thread pattern 780, the insert 778 may include a pilot hole (not shown) that the surgeon may tap to a desired size to match the thread pattern 782 of the pedicle fastener 710 or that the surgeon may install a self tapping pedicle fastener 710 within. In yet other embodiments, the insert 778 may not include any hole feature, but rather may allow the surgeon to both drill and tap the insert 778 to provide a hole of desired size and location on the insert. In some embodiments, the exterior surface of the insert 778 may itself be threaded to provide enhanced mechanical fixation within the hole 742. Preferably, the insert 778 is formed from substantially the same biocompatible polymeric material as that used to form the adhesive 728.

In operation, after preparation of the hole 742, the adhesive 728 may be applied to either the insert 778 or the hole 742 and then the insert 778 may be pushed into the hole 742. The adhesive 728 is then allowed to cure, bonding the insert 778 to the hole 742. The pedicle fastener 710 can then be threaded into the female thread pattern 780 or pilot hole (not shown) to affix a plate 783 or similar instrumentation thereto. The insert 778 advantageously allows a small pedicle fastener 710 to be screwed into a large bone hole 742. This may be particularly beneficial when the pedicle fastener 710 must pass through a plate 783 that limits the length of the shank 714. The insert 778 is also advantageous since it fills a majority of the hole 742 with a rigid volume that does not need to cure, whereas filling a similar hole 742 with only adhesive 728 would require significantly more curing time.

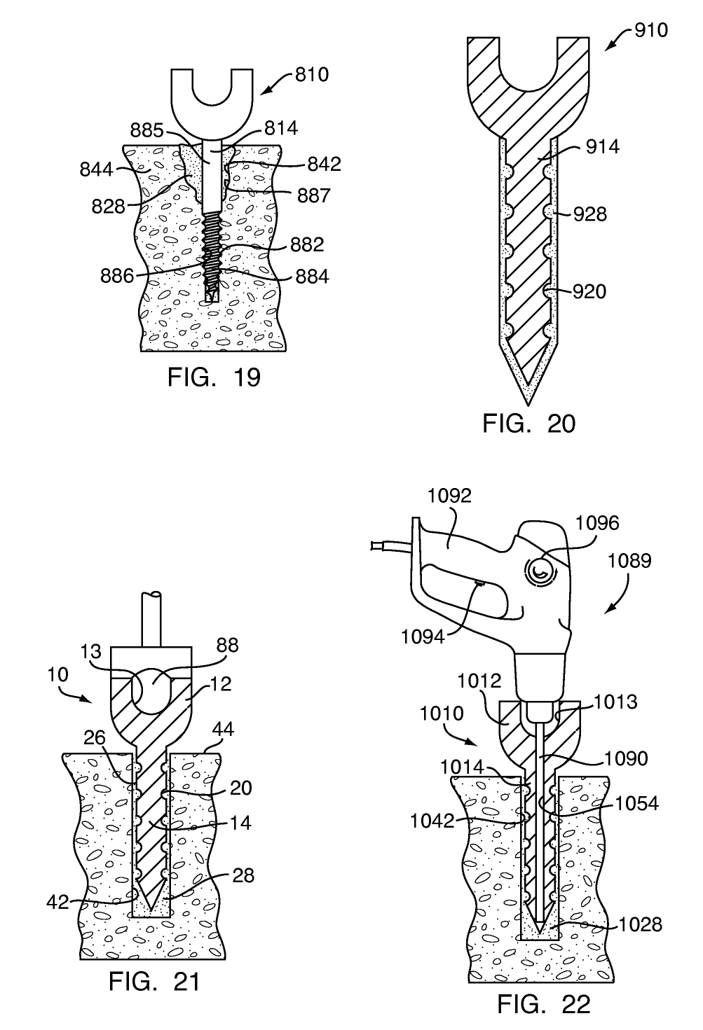

Referring to FIG. 19, in another embodiment of the present invention, the pedicle fastener 810 may include a shank 814 with a first longitudinal portion 884 having a thread pattern 882 and a second longitudinal portion 885 that is threadless. The pedicle fastener 810 may be advantageously applied to hole 842 having a first region 886 of good quality bone 844 and a second region 887 of poor quality bone 844, for example osteoporotic bone. In operation, the hole 842 may be prepared as discussed above and then adhesive 828 may be applied to the hole 842 and/or the shank 814 also as discussed above. The shank 814 may then be inserted into the hole 842, with the first longitudinal threaded portion 884 located in and threadedly engaged with the good quality bone 844 of the first region 886. The second longitudinal threadless region 885 may be located in the second region 887 where the bone 844 is of poor quality. Thus, the mechanical connection between the good quality bone 844 and the thread pattern 882 as well as the adhesive 828 secures and stabilizes the pedicle fastener 810 in the first region 886, while the pedicle fastener is secured in the second region 887 of poor quality bone 844 by the adhesive 828. As should be understood by those skilled in the art, the second longitudinal portion 885 of the shank 814 may be smooth or may include the various shielding features 18, shown in FIG. 1, discussed above. Additionally, as should also be understood by those skilled in the art, the location of the first longitudinal threaded portion 884 on the shank 814 may be varied to correspond to the first region 886 of good quality bone 844 and, in some embodiments, the shank 814 may include multiple longitudinal threaded portions 884 and/or multiple longitudinal threadless portions 885.

Referring back to FIG. 1, the embodiments discussed above have all included applying adhesive 28 to the pedicle fastener 10 or to the hole 42, shown in FIG. 6, substantially immediately prior to insertion of the shank 14 of the pedicle fastener 10 into the hole 42. However, referring to FIG. 20, in some embodiments of the present invention, the pedicle fastener 910 may be provided with frozen adhesive 928 coating the shank 914. In these embodiments, the adhesive 928 may be formed and applied to the shank 914 in substantially the same manner as discussed above. The pedicle fastener 910 may then be maintained at a decreased temperature to freeze the adhesive 928 to the shank and to prevent polymerization thereof. The pedicle fastener 910 may be stored in a sterile environment at the decreased temperature until needed for use in a spinal fusion procedure.

In operation, the pedicle fastener 910 with adhesive 928 frozen thereto may be pushed into the hole 42, shown in FIG. 4, preferably after preparation thereof, as discussed above. The patient's natural body temperature will elevate the temperature of the adhesive 928, which will cause the adhesive 928 to thaw within the hole 42, shown in FIG. 4, and polymerize therein forming the adhesive bond between the bone 44, shown in FIG. 4, and the shank 914. This embodiment may be particularly beneficial since it eliminates the necessity to prepare the adhesive 928 during the spinal fusion procedure. Although shown in connection with the pedicle fastener 910 having annular grooves 920, the frozen adhesive 928 may be equally applicable to the various other embodiments discussed herein. For example, in the embodiment having the mesh sleeve 70, shown in FIG. 17, connected to the shank 14, shown in FIG. 17, the adhesive 928 could be applied onto and into the mesh sleeve 70, shown in FIG. 17, and frozen therein for future use.

Although pre-application of the adhesive 928 has been described in connection with freezing the adhesive 928 to inhibit polymerization thereof, other methods for inhibiting polymerization of the adhesive 928 and pre-applying the adhesive to the shank 914 of the pedicle fastener 910 are also within the scope of the present invention. For example, in some embodiments, moisture curing adhesive 928, such as a one component moisture curing polyurethane, may be pre-applied to the shank 914 of the pedicle fastener 910 and then the pedicle fastener 910 may be packaged and/or stored in a moisture free environment until insertion into the hole 42, shown in FIG. 4 of the bone 44, shown in FIG. 4. Once inserted into the hole 42, shown in FIG. 4, moisture in the bone 44, shown in FIG. 4, would then activate the moisture curing adhesive 928 and allow the adhesive 928 to fully cure, thereby securing the pedicle fastener 910 within the bone 44, shown in FIG. 4.

In some embodiments of the present invention, it may be desirable to accelerate polymerization of the adhesive 28, shown in FIG. 6, most particularly in embodiments implementing the frozen adhesive 928 discussed above. Referring to FIG. 21, the pedicle fastener 10 may be provided with a removable heating extension 88 that connects thereto, for example, through connection feature 13 in substantially the same manner as a stabilizing rod or the like. The removable heating extension 88 is conductive, such that when heated, it conducts the heat to the pedicle fastener 10, which, in turn, heats up. The elevated temperature of the pedicle fastener 10 increases the temperature of the adhesive 10, which accelerates polymerization thereof. For example, an increase in temperature of the adhesive 28 of approximately ten degrees Celsius (10° C.) approximately doubles the polymerization rate of the adhesive 28. Once polymerization of the adhesive 28 is complete, the removable extension 88 may be removed from the head 12 of the pedicle fastener 10 and the pedicle fastener 10 may be attached to a stabilization rod or the like, as desired. In some embodiments, the removable heating extension 88 may be a break-away feature formed to be integral with the head 12 of the pedicle fastener 10. In these embodiments, once heating is completed, the removable heating extension 88 may simply be broken off of the head 12.

Referring to FIG. 22, in another embodiment, accelerated polymerization may be provided by a heating instrument 1089 having an electric heating element 1090 connected thereto. The heating instrument may include a handle 1092, an activation switch 1094 and a temperature gauge 1096. In some embodiments, the heating instrument 1089 may also include a temperature sensor that monitors and limits the peak temperature within the pedicle fastener 1010 to prevent excessive heating, which could damage surrounding tissue. The heating element 1090 may be long, slender and adapted to fit within the longitudinal cannula 1054 of the pedicle fastener 1010. Preferably, various heating elements 1090 having varying diameters may be connectable to the heating instrument 1089 to provide functionality with different pedicle fasteners 1010 having different sized longitudinal cannulas 1054 formed therein. These heating elements may be formed from nichrome or the like. In some embodiments, at least a portion of the heating element 1090 may be adapted to simply rest in the stabilization rod connection feature 1013 of the head 1012 or may be adapted to be accommodated over or around a portion of the head 1012.

In operation, once the pedicle fastener 1010 has been inserted into the hole 1042, as discussed above, the heating element 1090 of the heating instrument 1089 may be inserted into the longitudinal cannula 1054 or may be brought into contact with the connection feature 1013 or a portion of the head 1012. The surgeon may then activate the heating instrument using the activation switch 1094. As the temperature of the heating element 1090 increases, heat is transferred through the shank 1014 and into the adhesive 1028. As discussed above, the increase in temperature of the adhesive 1028 increases the polymerization rate thereof. Once polymerization of the adhesive 1028 is complete, the surgeon may deactivate the activation switch 1094 removed the heating element 1090 from the pedicle fastener 1010.

In addition to the electrical and/or conductive heating discussed above for accelerating polymerization of the adhesive 1028, various other forms of heating, i.e. radiant, chemical, frequency or other forms of conductive heating, may be used to heat the pedicle fastener 1010 and accelerate polymerization of the adhesive 1028. For example, in some embodiments, a heat lamp may be position to direct radiant heat toward the spinal fusion site and the pedicle fastener 1010. In some embodiments, the temperature of the pedicle fastener 1010 may be elevated prior to implantation, for example, in an oven, in hot water or the like, such that when inserted into the adhesive 1028, the heat stored in the pedicle fastener 1010 accelerates polymerization of the adhesive 1028. This embodiment may be particularly beneficial since it does not require a heat source that connects to the pedicle fastener 1010.

The present invention advantageously provides various pedicle fasteners 10, 110, 210, 310, 410, 510, 610, 710, 810, 910 and 1010 and methods for implantation thereof that allow augmentation with adhesive 28, 128, 528, 628, 728, 828, 928 and 1028 without removal of the adhesive 28, 128, 528, 628, 728, 828, 928 and 1028 from the shank 14, 114, 214, 314, 414, 514, 614, 714, 814, 914 and 1014 of the pedicle fastener 10, 110, 210, 310, 410, 510, 610, 710, 810, 910 and 1010 during insertion. The shank 14, 114, 214, 314, 414, 514, 614, 714, 814, 914 and 1014 of the pedicle fastener 10, 110, 210, 310, 410, 510, 610, 710, 810, 910 and 1010 may advantageously be provided with various adhesive shielding features 18, 218, 318 and 518 and attachments to allow adhesive migration and expansion, where desired, while preventing unwanted migration and expansion. Additionally, the shank 14, 114, 214, 314, 414, 514, 614, 714, 814, 914 and 1014 may be connectable to known pedicle fastener heads 12, 412, 512 and 612 to provide interchangeability thereof.

Another advantage of the present invention is that it provides pedicle fasteners 10, 110, 210, 310, 410, 510, 610, 710, 810, 910 and 1010 having non-polymerized adhesive 28, 128, 528, 628, 728, 828, 928 and 1028 frozen thereto for direct implantation in the pedicle hole 42, 142, 542, 642, 742, 842 and 1042. This advantageously eliminates the need to form the adhesive 28, 128, 528, 628, 728, 828, 928 and 1028 during the spinal fusion procedure, which may reduce the complexity of the procedure as well as the operating room time for performing the spinal fusion procedure.

A further advantage of the present invention is that it provides methods and devices for accelerating polymerization of the implanted adhesive 28, 128, 528, 628, 728, 828, 928 and 1028, which can reduce operating room time for performing spinal fusion procedures. Additionally, the methods and devices for accelerated polymerization may be particularly beneficial for accelerating polymerization in connection with pedicle fasteners 10, 110, 210, 310, 410, 510, 610, 710, 810, 910 and 1010 having frozen adhesive 28, 128, 528, 628, 728, 828, 928 and 1028 deposited thereon.

A further advantage of the present invention is that the pedicle fastener 10, 110, 210, 310, 410, 510, 610, 710, 810, 910 and 1010 is compatible with known stabilization instrumentation, meaning that no customization is required for spinal fusion procedures.

Although this invention has been shown and described with respect to the detailed embodiments thereof, it will be understood by those skilled in the art that various changes in form and detail thereof may be made without departing from the spirit and the scope of the invention. For example, although the pedicle fasteners have been described for providing stabilization after spinal fusion surgery, the pedicle fasteners may also be configured for other orthopedic applications such as fusion of critical defects in other bones.

What is claimed is:

1. A spinal fusion kit comprising:
at least one pedicle fastener having a head and a shank, the shank extending from the head to a shank tip, and having a surface suitable for receiving a biocompatible polymeric material; and
a biocompatible polymeric material for augmenting the at least one pedicle fastener.

2. The spinal fusion kit according to claim 1, wherein the shank has at least one shielding feature formed therein.

3. The spinal fusion kit according to claim 2, wherein the at least one shielding feature includes at least one annular groove extending around a circumference of the shank.

4. The spinal fusion kit according to claim 3, wherein the shank comprises a plurality of annular grooves extending around the circumference of the shank.

5. The spinal fusion kit according to claim 2, wherein the at least one shielding feature includes at least one flute extending longitudinally along the outer surface of the shank.

6. The spinal fusion kit according to claim 5, wherein the at least one flute extends over substantially an entire length of the shank.

7. The spinal fusion kit according to claim 5, wherein the shank comprises a plurality of flutes extending longitudinally along the outer surface.

8. The spinal fusion kit according to claim 7, additionally comprising a plurality of annular grooves extending around the circumference of the shank,
wherein the plurality of annular grooves are in communication with the plurality of flutes.

9. The spinal fusion kit according to claim 2, wherein the at least one shielding feature includes a spiral flute.

10. The spinal fusion kit according to claim 2, wherein the at least one shielding feature includes threads.

11. The spinal fusion kit according to claim 1, wherein the shank comprises a longitudinal slot extending substantially the entire length of the shank, the longitudinal slot being in communication with a longitudinal channel within the shank, the longitudinal channel being substantially coaxial with the shank.

12. The spinal fusion kit according to claim 1, wherein the biocompatible polymeric material is provided on at least a portion of the shank of the pedicle fastener.

13. The spinal fusion kit according to claim 12, wherein the biocompatible polymeric material is frozen.

14. The spinal fusion kit according to claim 1, wherein the at least one pedicle fastener includes a heating feature for applying a heat source to the pedicle fastener.

15. The spinal fusion kit according to claim 14, wherein the heating feature includes a removable extension extending from the head of the pedicle fastener.

16. The spinal fusion kit according to claim 1, wherein the shank tip includes a reverse taper.

17. The spinal fusion kit according to claim 1, wherein the pedicle fastener includes a longitudinal cannula extending at least partially through the head or shank.

18. The spinal fusion kit according to claim 17, wherein the pedicle fastener additionally includes at least one fenestration connecting the longitudinal cannula with at least one shielding feature formed in the shank.

19. The spinal fusion kit according to claim 1, wherein the pedicle fastener additionally includes at least one restrictive disc attached to the shank.

20. The spinal fusion kit according to claim 1, wherein the shank is polyaxial relative to the head.

21. The spinal fusion kit according to claim 1, additionally comprising at least one insert having a female thread pattern formed therein;
wherein the pedicle fastener includes threads formed on the shank that match the thread pattern of the insert.

22. The spinal fusion kit according to claim 1, wherein the at least one pedicle fastener additionally includes a surface coating formed on the shank.

23. The spinal fusion kit according to claim 22, wherein the surface coating is formed from substantially the same material as the biocompatible polymeric material for augmenting the at least one pedicle fastener.

24. The spinal fusion kit according to claim 1, additionally comprising a mesh sleeve having a longitudinal opening at one end thereof adapted to accept the shank of the at least one pedicle fastener.

25. The spinal fusion kit according to claim 24, wherein the mesh sleeve surrounds the shank and is connected thereto.

26. The spinal fusion kit according to claim 1, wherein the biocompatible polymeric material is polyurethane.

27. A pedicle fastener comprising:
a head; and
a shank, the shank extending from the head to a shank tip, the shank having at least one nonthreaded shielding feature formed therein.

28. The pedicle fastener according to claim 27, wherein the at least one shielding feature includes at least one annular groove extending around a circumference of the shank.

29. The pedicle fastener according to claim 28, wherein the shank comprises a plurality of annular grooves extending around the circumference of the shank.

30. The pedicle fastener according to claim 27, wherein the at least one shielding feature includes at least one flute extending longitudinally along the outer surface of the shank.

31. The pedicle fastener according to claim 30, wherein the at least one flute extends over substantially an entire length of the shank.

32. The pedicle fastener according to claim 30, wherein the shank comprises a plurality of flutes extending longitudinally along the outer surface.

33. The pedicle fastener according to claim 32, additionally comprising a plurality of annular grooves extending around the circumference of the shank,
wherein the plurality of annular grooves are in communication with the plurality of flutes.

34. The pedicle fastener according to claim 27, wherein the shank comprises a longitudinal slot extending substantially the entire length of the shank, the longitudinal slot being in communication with a longitudinal channel formed within the shank, the longitudinal channel being substantially coaxial with the shank.

35. The pedicle fastener according to claim 27, wherein the at least one shielding feature includes a spiral flute.

36. The pedicle fastener according to claim 27, additionally comprising a frozen biocompatible polymeric material provided on at least a portion of the shank.

37. The pedicle fastener according to claim 27, additionally comprising a heating feature for applying a heat source to the pedicle fastener.

38. The pedicle fastener according to claim 37, wherein the heating feature includes a removable extension extending from the head of the pedicle fastener.

39. The pedicle fastener according to claim 27, wherein the shank tip includes a reverse taper.

40. The pedicle fastener according to claim 27, additionally comprising a longitudinal cannula extending at least partially through the head or shank.

41. The pedicle fastener according to claim 40, additionally comprising at least one fenestration connecting the longitudinal cannula with the at least one shielding feature.

42. The pedicle fastener according to claim 27, additionally comprising at least one restrictive disc attached to the shank.

43. The pedicle fastener according to claim 27, wherein the shank is detachable from the head.

44. The pedicle fastener according to claim 27, additionally comprising a surface coating formed on at least a portion of the shank.

45. The pedicle fastener according to claim 44, wherein the surface coating is formed from a biocompatible polymeric material.

46. The pedicle fastener according to claim 27, additionally comprising a mesh sleeve having a longitudinal opening at one end thereof, the mesh sleeve surrounding the shank and connected thereto.

47. A method for implanting a pedicle fastener having a head and a shank, the method comprising:
forming a hole in a pedicle having a diameter sufficient to receive the shank of the pedicle fastener;
applying an adhesive to the hole; and
positioning the shank of the pedicle fastener in the hole.

48. The method according to claim 47, additionally comprising applying the adhesive to the shank of the pedicle fastener;
wherein the adhesive is applied to the hole when the shank is positioned therein.

49. The method according to claim 48, wherein applying the adhesive to the shank includes applying the adhesive to at least one shielding feature formed therein.

50. The method according to claim 49, wherein the at least one shielding feature includes a least one annular groove.

51. The method according to claim 49, wherein the at least one shielding feature includes at least one longitudinal flute.

52. The method according to claim 47, wherein applying the adhesive to the hole includes injecting the adhesive through a syringe.

53. The method according to claim 47, wherein applying the adhesive to the hole includes injecting the adhesive through a longitudinal cannula of the pedicle fastener.

54. The method according to claim 47, additionally comprising forming the adhesive by mixing a prepolymer component, a polyol component and a filler material.

55. The method according to claim 47, additionally comprising heating the pedicle fastener to accelerate polymerization of the adhesive.

56. The method according to claim 55, additionally comprising connecting a heating element to the head of the pedicle fastener.

57. The method according to claim 55, additionally comprising inserting a heating element into a longitudinal cannula extending through the head and the shank of the pedicle fastener.

58. The method according to claim 55, wherein the pedicle fastener is heated with a heat lamp.

59. The method according to claim 55, wherein the pedicle fastener is heated prior to insertion of the shank into the hole.

60. The method according to claim 47, additionally comprising allowing the adhesive to expand into surrounding bone of the pedicle after the pedicle fastener has been seated in the hole.
